(19) United States
(12) Patent Application Publication
(10) Pub. No.: US 2023/0051604 A1
(43) Pub. Date: Feb. 16, 2023

(54) HANDHELD COLD PLASMA DEVICE

(71) Applicant: Thi Hong Lien Luong, Hanoi (VN)

(72) Inventor: Thi Hong Lien Luong, Hanoi (VN)

(21) Appl. No.: 17/759,216

(22) PCT Filed: Jan. 20, 2021
(86) PCT No.: PCT/VN2021/000003

§ 371 Date: Jul. 21, 2022

(30) Foreign Application Priority Data
Jan. 21, 2020 (VN) 1-2020-00428

(51) Int. Cl. A61B 18/12; H05H 1/24

(52) U.S. Cl. CPC A61B 18/1206; H05H 1/2418; A61B 2018/00452

(57) ABSTRACT

A handheld cold plasma device in which the device includes power source, control electronic circuit boards connected to high-frequency high-voltage transformers, user control panels, insulating case covering control electronic circuits, high-frequency high-voltage transformer and user control panel to form a monoblock, the plasma generating unit contains the active electrode and the passive electrode outside the housing to allow the user to be connected to the neutral wires of the device. The device according to the present disclosure uses the principle of direct discharge (or dielectric barrier discharge with floating electrodes) with small size, inexpensive and does not use consumable materials.

### CROSS-REFERENCE TO RELATED APPLICATIONS

This application is a national phase entry under 35 U.S.C. § 371 of International Patent Application PCT/VN2021/000003, filed Jan. 20, 2021, designating the United States of America and published as International Patent Publication WO 2021/151124 A1 on Jul. 29, 2021, which claims the benefit under Article 8 of the Patent Cooperation Treaty to Vietnamese Patent Application Serial No. 1-2020-00428, filed Jan. 21, 2020.

### TECHNICAL FIELD

The present disclosure relates to a device generating cold plasma used for therapeutic treatments. Specifically, the present disclosure relates to a cold plasma device using the principle of direct discharge or discharge through the dielectric-barrier with floating electrodes, also known as direct plasma or plasma FE-DBD (Floating Electrode Dielectric-barrier discharge). The plasma is generated between the generating unit and the surface to be treated, so the device is compact in size, inexpensive, and doesn't use consumable materials.

### BACKGROUND

The application of cold plasma to disinfect and promote the wound healing has become very popular. However, nowadays, most of cold plasma devices use gas exchangers, so their structure is complex and cumbersome.

Currently, on the market, there have been some handheld plasma device, however these devices are often overconsuming, and do not optimize the energy of the plasma stream, causing pain to patients.

Handheld plasma device with the trade name “The plasma Care” use the principle of surface micro discharge. The principle involves an electrical discharge between two mesh electrodes without dielectric barrier so that the electrical impulses have relatively high energy, which requires ensuring there is enough distance between the pair of electrodes and the surface to be treated to avoid electrical discharges directly to avoid injury. For that reason, the device must have air flow through the plasma forming region to bring plasma to the wound, which is why the effect of the plasma is not optimal. In addition, the structure of this device is only capable of being used for the treatment of open wounds.

The device with the trade name “plasma ONE” uses the principle of direct discharge through the dielectric barrier with an inert gas electrode. This device generates a very disturbed and relatively strong electrical pulse. The plasma energy is mediated by the plasma lamp inside the main electrode. This does not optimize the energy transmitted by the pulse, causing pain when in contact with open wounds. In addition, the device does not have the ability to produce polarized electrical pulses.

The device with the trade name “plasmaderm” uses the principle of floating electrodes dielectric barrier discharge. This device uses alternating current with high voltage and low oscillation frequency (50 Hz) to form plasma, causing pain when exposed to open wounds. The distance between the main electrode and the surface of the wound to be treated is ensured by a static plots structure with a relatively large area, but the uniformity of plasma energy over the treated area is not guaranteed.

The device under the trade name “Plasma Shower” uses the principle of floating electrodes dielectric barrier discharge. However, this device does not have a ground to stabilize the voltage, so the operation of the device is unstable. In addition, this device uses alternating current with low voltage to avoid harm, so it also greatly affects the stability and efficiency of plasma formation.

The device with the trade name “MIRARI” uses the principle of non-direct dielectric barrier discharge using a pair of grid electrodes separated by a dielectric barrier. The plasma generated between these two electrodes will spread out to the area of the wound that needs treatment. However, this device does not optimize the energy generated by the pulse.

Recent studies have shown that using the direct dielectric barrier discharge with floating electrodes has a much higher therapeutic (bactericidal) effect than the gas exchange principle (or plasma jet) (see diagrams comparing direct plasma versus plasma jet in bactericidal tests as shown in FIGS. 1 to 3).

In the principle of direct plasma, unlike the indirect principles, the patient's body acts as the second electrode of the electrode pair, so the total of plasma current goes through the patient's body. This electric current has a stimulating effect that accelerates wound healing.

However, the plasma direct principle causes a strong or light biting sensation depending on whether the plasma streams are large or small.

In the patent application WO2019121968, the effect of large plasma streams is used to create intracellular/extracellular micro-holes, to aid in drug osmosis. However, in order to treat a wound, it is necessary to minimize these streams to relieve the patient pain.

The patent application WO/1999/043782 and US 2005/0177092 A1 disclosed that the monopolar electrical pulses of plasma generation create “electrophoresis” effect on cells to help drug osmosis.

However, these disclosures are interested in only the polarization of electrical impulses (electrical impulses are always negative or always positive), and not in the polarization of the plasma (which produces positive or negative streams).

Therefore, there is a need for a direct plasma-beam device that has a compact structure, is capable of optimizing the pulse energy and not causing pain for the patient, and at the same time, is capable of switching between positive and negative plasma according to the request of the user.

### BRIEF SUMMARY

It's an objective of the present disclosure to provide a handheld cold plasma device that serves a wide variety of clinical applications and has the potential to be used by the patient himself The device is capable of creating a uniform cold plasma, avoiding pain, ensuring safety, stability and ease of use and having a low maintenance cost.

The plasma device of the present disclosure includes a power source (e.g., rechargeable battery), electronic circuit board, user control panel, insulating case, plasma generating unit and auxiliary electrode outside the insulating case connected to electronic circuits thanks to capacitors.

According to the present disclosure, the plasma device also includes a high-frequency high-voltage transformer that produces a damped sinusoidal pulse.

The plasma device of the present disclosure is programmed to be suitable for various applications such as: disinfection of open wounds, abrasions, postoperative wounds, chronic wounds, burns and treatment of skin disorder and diseases, oral hygiene or gynecological hygiene as recommended by a doctor. There is also an antiseptic aid for open or endoscopic surgeries.

The device of the present disclosure can also be connected to a smart device such as a smartphone, smart watch, computer, etc., to exchange information related to the protocol of treatment.

### BRIEF DESCRIPTION OF THE DRAWINGS

FIG. 1 shows a killing test of P. aeruginosa bacteria on agar using a direct DBD plasma (left) and a plasma jet (right).

FIG. 2 shows the killing time of P. aeruginosa bacteria in a solution using direct DBD plasma and plasma jet.

FIG. 3 is a diagram showing bacteria killing time on damaged and healed skin using direct DBD plasma and plasma jet.

FIG. 4 is a schematic representation of a damped sinusoidal electric pulse emitted by a high-frequency high-voltage transformer in the device according to the present disclosure.

FIG. 5 is a schematic illustration showing the principle of the Ruhmkorff ferromagnetic resonance.

FIG. 6 is a schematic representation of two or more consecutive electrical impulses forming a series.

FIG. 7 shows a structural diagram of the transformer according to one embodiment of the present disclosure.

FIG. 8 shows the internal structure of a plasma device according to the present disclosure.

FIG. 9 shows the structure of the capacitor connecting the auxiliary electrode to the ground circuit.

FIGS. 10 and 11 show different plasma generating unit that can be attached to the device according to the present disclosure.

FIG. 12 shows the results of microbiological testing of device according to the present disclosure.

FIG. 13 shows images of the test results on animals.

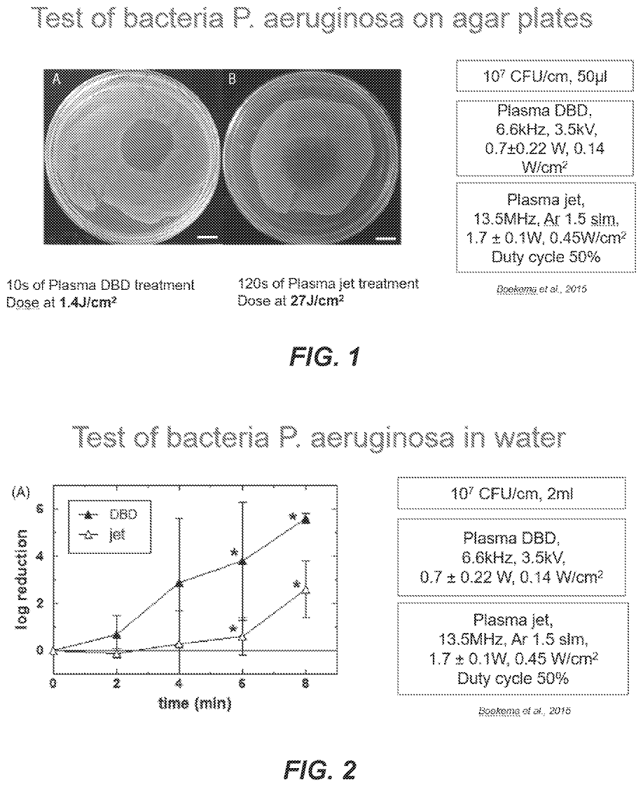

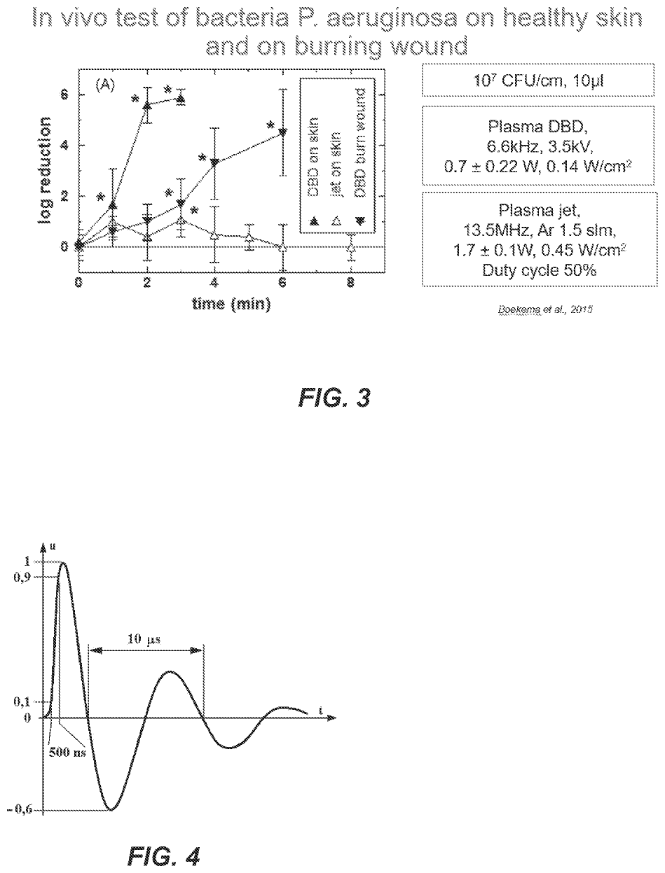

### DETAILED DESCRIPTION

The following detailed description is provided to help the reader in gaining a comprehensive understanding of the equipment and the method described herein. The various parameters, variations and equivalents of the devices and methods described herein will be apparent to others skilled in the art.

It should be noted that the terms used in the present disclosure are not intended to limit the present disclosure but are used only to allow a clear and consistent understanding of the present disclosure.

Accordingly, it is apparent to others skilled in the art that the following description of the present disclosure is provided for the illustrative purposes only and is not intended to limit the invention as defined by the accompanying claims and their equivalents.

A cold plasma handheld device according to the present disclosure comprises:
Power source
Control electronic circuit board provides low voltage pulse to the high-frequency high-voltage transformer
High-frequency high-voltage transformer
User control panel
Insulating case with an opening for connecting with the plasma generating unit and opening for connecting with the power source
Active electrodes on the plasma generating unit
Passive electrodes outside the housing of the device.

### Power Source

The device's power source can be either a normal battery, a rechargeable battery or any other power source that gives the electronic circuit board direct current.

### Electronic Control Circuit Board

The basic feature of the control circuit board is to convert direct current from the power source to a series of electrical pulses transmitted to a high-frequency high-voltage transformer according to predetermined programs with pre-programmed pulse parameters in the circuit microcontroller/microprocessor. The control electronic circuit receives control commands and pulse parameters from the user control panel and/or from the smart device connected to the control electronic circuit through the IoT connectivity.

In addition to being able to change the polarization sign of the plasma by changing the polarization sign of the electrical pulse applied to the transformer, it is also possible to change the pulse frequency to adjust the density of the resulting plasma or its successive pulses to increase plasma stream size as needed. The electronic circuit controls the plasma generating time, thereby controlling the plasma dose for each treatment. With sound (beep) and/or vibrating signaling systems, the user is always informed of the dose of plasma used. The device can be programmed to automatically turn off when the dose of the emitted plasma is optimal for user safety.

The control circuit can integrate an Internet of Things (IoT) connection to exchange information with smart devices such as smart phones, smart watches, computers, etc., via wired communication (e.g., USB), or wireless communication such as Wi-Fi or RFID or BLUETOOTH® or NFC. In addition to information transmitted to smart device application software for treatment management, this communication also allows the smart device application software to program the plasma device according to new, specific parameters of treatment when needed.

The effectiveness of plasma treatment is very influenced by the dose of plasma used per treatment as well as the frequency of use. When plasma dosage is increased, the effect can range from bactericidal, proliferating, eliminating cancer cells to necrosis, burns, etc. When using plasma too often, it can cause dry skin, disorder skin's microbiotat, etc. It is therefore very important to control plasma dosage and frequency for self-treatment. In the case of the patient being treated at home, or in the case of using virtual doctor technology, the use of smart devices to identify the patient (by keywords, fingerprints, etc.) to store the patient records, doctor's prescription and treatment plan help strictly control the treatment protocol to avoid abuse or forgetting to use plasma.

In addition, the control circuit also has the function of managing the charge, controlling the remaining power level, ensuring the thermal safety, etc., of the battery.

### High-Frequency Voltage-Transformer

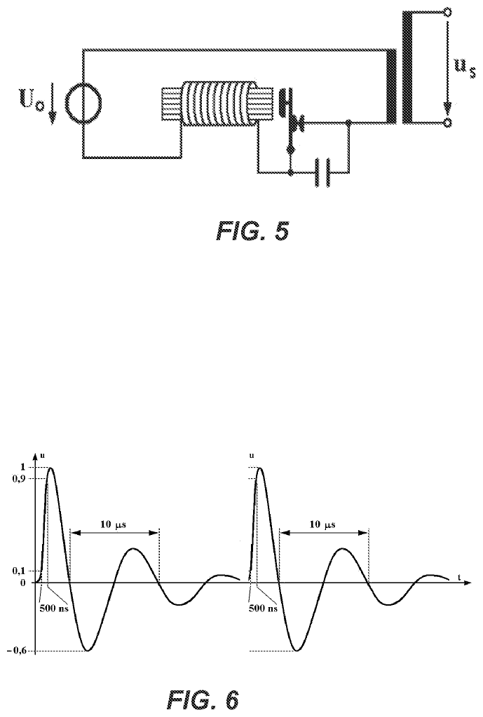

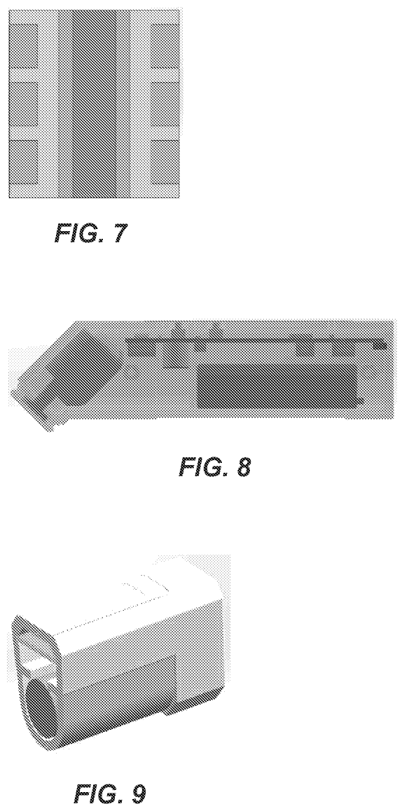

The elementary electrical pulse that produces a series of electrical impulses generated by the high-frequency high-voltage transformer is a damped sinusoidal pulse. The characteristic of this pulse is that the first half sinusoidal has a high amplitude but a short width while the second half sinusoidal has a smaller amplitude but is wider to create capacitive balance (FIG. 4). The polarity sign of the plasma stream depends on the polarity sign of the first half sinusoidal. The shape of such electrical pulses empowers the activation of the plasma's formation from air with as little energy as possible while still producing a mixture of highly efficient reactive ingredients for therapeutic purposes and reducing the patient's pain. With this electrical pulse, the plasma device can convert an energy as low as a few Watts (W), very safely while creating an efficient plasma.

The oscillation frequency of the sine wave is calculated to match a frequency that consistently forms in the air a plasma (above 10 kHz) with tiny filaments (no more than a few tens of micrometers). The oscillation frequency of the sine wave also depends on the output voltage, the distance between the electrodes, etc. An example of an application in a handheld cold plasma device according to the present disclosure is to have an oscillation frequency of 100 kHz with a margin the highest pulse voltage about 6 kV to create a uniform plasma within the distance between the electrodes, which may be less than 1 mm.

There are many principles of high-frequency high-voltage transformers that can be used to generate high-voltage pulses. The Ruhmkorff ferromagnetic resonance principle is one of the simplest and most effective principles for generating such a pulse waveform with the primary input pulse being just a square electrical pulse, which is common in electronic circuits. The polarity sign of the generated plasma depends on the sign of the electrical impulse applied to the primary coil.

This principle has the advantage of being able to convert to a very high voltage in the secondary coil, which can be up to thousands of times compared to the input voltage of the primary coil. The design of this transformer is characterized by two coaxial primary and secondary coils on a ferromagnetic core. The secondary coil is rolled into several parts separated by insulated barrier to ensure that no discharge occurs inside the coil.

The oscillation frequency of the pulse generated at the secondary stage depends on the L and R parameters of the coil.

### User Panel

The user panel includes buttons, LED indicators, display, fingerprint sensor, and more. The user panel allows the device user to select parameters related to treatments, turn on/off the device, control the power condition, etc.

### Plasma Generating Unit

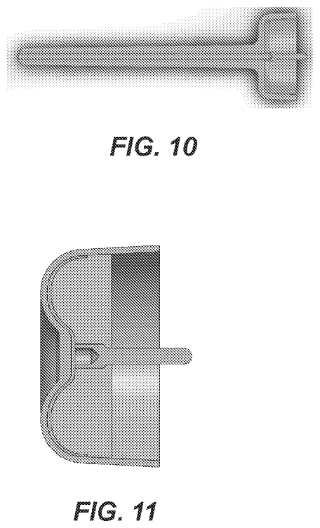

The plasma generating unit is where plasma is generated when it receives energy from a high-frequency high-voltage transformer and when it comes near or in contact with the surface to be treated. The main components of the plasma generating unit are active electrodes and surrounding insulation. The plasma generating unit in the device of the present disclosure is a detachable accessory. In addition to the active electrode and insulation layer, the plasma generating unit also has an electrical connector that allows it to connect to the output of the high-frequency high-voltage transformer and allows it to be easily removable by the user. The device according to the present disclosure can be connected to a variety of plasma generating units depending to the desired application, such as a direct plasma generating unit (FIG. 10), or a direct DBD plasma generating unit (FIG. 11).

### Passive Electrode

The passive electrode is a thin layer of conductive material that partially covers the handle area outside the insulating case, and inside this insulator part, a second layer of conductive material parallel to the passive electrode to forms a capacitor. It is this second layer of conductive material that is connected to the ground (or mass wire) of the circuit board or the primary transformer coil. As the example is shown in FIG. 9, the second layer of conductive material is the battery cover, inside the device.

### EXAMPLES

For different applications, different treatment areas, different treatment protocols will be available.

### Example of Application Number 1

One of the applications of the device under the present disclosure is to generate a series of pulses with pulses frequency as high as several tens of kHz (the interval between each pulse is some tens of microseconds) in a short period of time. This sequence of pulses will generate and sustain the plasma stream for a period long enough to have enough energy to burn the cell. The interval between two successive sequences of a few hundred microseconds helps to interrupt the plasma stream. The effect of this series of pulses is to create very small burning points on the skin causing anti-aging, firm and tightening effects. The plasma generating unit used for this therapy is usually the direct discharge plasma generating unit as shown in FIG. 10.

Adjusting the length of each sequence of pulses affects the depth of the burning point and causes a tingling feeling within the limits that can be tolerated. The number of sequences emitted at each burning point affects the width of the burner. These parameters can be controlled via the user control panel that affects the control circuitry. Usually, the energy of each sequence should not exceed 100 mJ to avoid pain.

The therapy can also be used to burn deep tissue inflammatory sites such as granulomatosis, HSV, gonorrhea, etc.

This treatment can be combined with healing therapy (e.g., application 3) so that the burning points can heal quickly.

### Example of Application Number 2

In applications for sterilization, anti-viral, fungus treatment on tissue and skin, the applicator used will be the direct DBD type.

The plasma applicator consists of an electrode of a certain size surrounded by a dielectric layer as shown in FIG. 11.

The key parameters for the treatment programming will be those related to the plasma dose per unit area (J/cm²); plasma power density (W/cm²), which affects the tolerable permeability and the polarity of the plasma (negative/positive), which affects the desired biological effect.

In this case, electrical pulses will be discharged regularly with the desired wave polarity, and the pulse frequency is adjusted to adjust the plasma power density. Plasma dosage will be calculated based on density and duration of plasma treatment per unit area to be treated. In general, for safety reasons, the plasma power produces no more than a few Watts with a density below 1 W/cm²; preferably the density should not exceed 0.3 W/cm²

Through the specific application software that allows calculating the area, the gravity of infection, from which to give optimal treatment parameters, the device can be programmed automatically according to the treatment protocol based on imaging diagnosis (acne, atopic dermatitis, eczema, etc.), infected areas. By applying artificial intelligence (AI), the software can make not only accurate diagnosis conclusions but also optimize the plasma parameters needed for treatment.

### Example of Application Number 3

In the treatment of infected wounds, acute or chronic, in addition to anti-inflammatory and antiseptic effects, plasma also has a hemostatic effect, stimulates cell proliferation, epithelialization, microvascular, etc., heal faster. Depending on the condition of the wound, the dose of plasma per treatment and the number of treatments per week must be calculated to match the level of infection, gangrene and healing stages.

The use of AI in this application will help diagnose and indicate wound treatment through image processing and update medical file/record to help make the right treatment for each patient depending on their recovery ability.

On the wound surface that is still too wet, the use of a thin layer of gauze to cover the wound surface during the treatment process makes the movement of the plasma generating unit easier, cleaner without affecting the performance of plasma treatment. The gauze should be as thin and breathable as possible and the material of the gauze must have a neutral or negative electrostatic coefficient.

### Advantageous Effects of the Present Disclosure

The device according to the present disclosure utilizes the dielectric barrier discharge principle with a floating electrode (or direct plasma) is one of the systems that does not use gas exchange. The device according to the present disclosure has the advantage of being very compact, convenient, inexpensive and does not use consumable materials (inert gas, etc.).

The use of a capacitor structure to connect to the ground gives the device flexibility, without messy cables, and ensures a minimal reduction of impedance for electrical impulses while still being safe compared to massive resistive systems. The use of a capacitor structure also increases plasma forming efficiency.

The device is capable of producing polarized plasma and can reverse electrical impulses to change the polarity of the generated plasma to suit treatment needs.

The device creates a cold plasma evenly, which avoids the tingling sensation of the patient, and ensures safety, stability and ease of use.

Test results of cold plasma equipment according to the present disclosure:

### Microbiological Testing on Agar Plates (In Vitro)

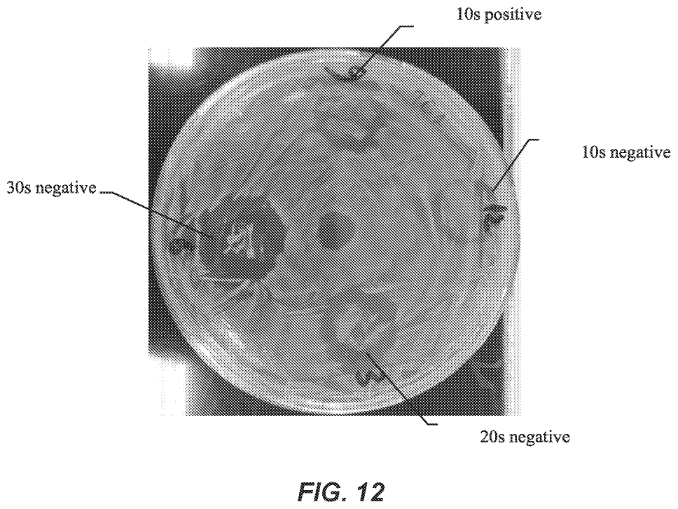

Microbiological study with the code N° M1HFP01B01VNM-MBIP using cold plasma device model HFP01 according to the present disclosure, with the plasma generating unit as shown in FIG. 11, was done by the National Institute of Hygiene and Epidemiology for the purpose of evaluating the ability to kill microorganisms of the device at 3 different time levels 10 seconds, 20 seconds, 30 seconds (energy equivalent 2.3 J/cm²; 4.6 J/cm²; 6.9 J/cm², respectively) on strains of MRSA resistant Staphylococus aureus (examples in FIG. 12), Pseudomonas aeruginosa, Escherichie coli, Enterobacter faecalis, Bacillus cereus, Candida albicans on agar plates showed:

1—Kill microorganisms reaching over 99.99% from the lowest energy level of 2.3 J/cm² (10 seconds).

2—The killing effect increases (the area of the microorganism-destroyed area) when the plasma dose increases.

3—The effect of killing Pseudomonas aeruginosa and Bacillus cereus of two positive and negative plasma modes is similar.

4—The effectiveness of negative plasma in killing Staphylococcus aureus, Escherichia coli, Enterobacter faecalis, Candida albicans is higher than that of the positive mode of plasma.

5—The device can also kill microorganisms that are difficult to destroy such as spore-forming bacteria, fungi, and antibiotic-resistant bacteria.

### Pre-Clinical Trials on Animals

Research conducted by the pharmacology department of Hanoi Medical University with the aim of evaluating the treatment effects of gangrene burns and systemic effects of cold plasma therapy with HFP device according to the present disclosure on Wistar rats with two daily therapeutic doses of 5 J/cm² and 10 J/cm². The conclusion of this study shows:

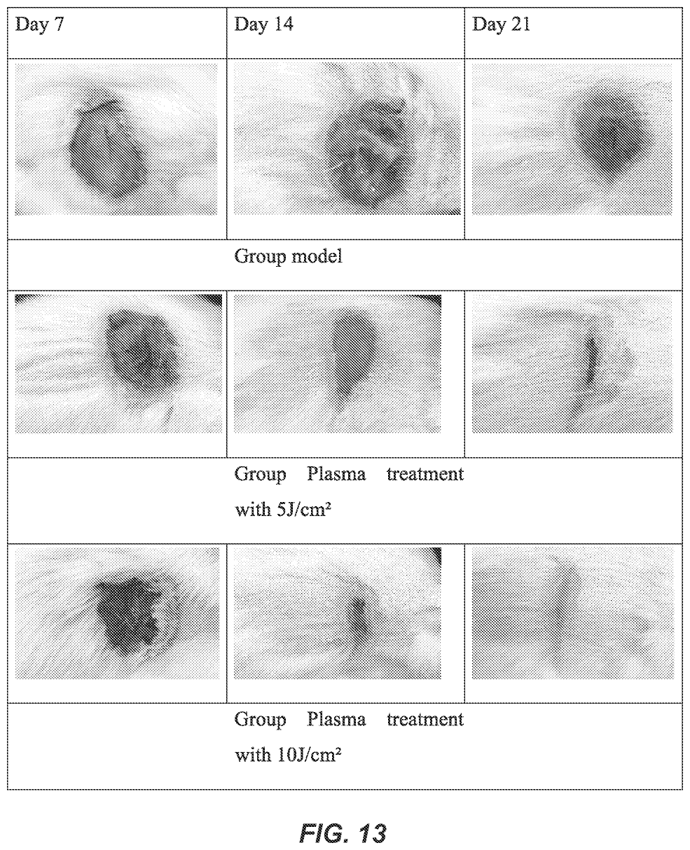

1—Only after 1 week of treatment, plasma therapy groups showed signs of shrinking the burn area compared to the model lot and after 3 weeks, the difference was statistically significant (p<0.05) as shown in FIG. 13. The group with 10 J/cm² dose showed the most rapid effect.

2—Quantitative analysis of hydroxyproline concentration in damaged tissue compared to healthy skin after 21 days of the model lot reduced significantly (13.18±4.72 mg/g compared to 26.84±8.99 mg/g), statistical signification (p<0.01), on the contrary, both plasma therapy groups gave similar good results (24.32±8.15 mg/g for the group with 5 J/cm² and 24.82±8, 29 mg/g for group with 10 J/cm²) to healthy skin.

3—After 21 days there is a clear difference in the microscopic morphological structure of the burn area between the model group and the plasma HFP therapy groups. In the burns area of rats in the model group, the epidermis covered little, with obvious burn lesions, the epidermis and skin-dependent glands disappeared, and there were severe inflammatory lesions and lots of inflammatory cells. At the burns area of the groups of rats receiving HFP plasma therapy with both doses: The epidermal burn area covered widely, small lesions left. Many regions have good regeneration, many new blood vessels. Fewer inflammatory cells. The plasma HFP therapy group at 10 J/cm² had more newly regenerated epidermal regions than the treatment group at 5 J/cm².

Systemic effects of plasma HFP therapy on experimental burn modeling showed:

1—Both plasma therapeutic doses did not affect the general condition as well as the degree of weight gain of the rats compared with those in the control group.

2—Do not change the results of tests assessing hematopoietic function (number of red blood cells, hemoglobin content, hematocrit, average volume of red blood cells, number of leukocytes, white blood cell formula, number platelets) compared with the control group.

3—No changes in the results of tests to assess liver function (total bilirubin, albumin and total cholesterol in rat's blood) compared to the control group.

4—No damage to liver cells (AST, ALT activity in rat's blood) compared to the control group.

5—Do not change the results of creatinine test in the blood of rats after 21 consecutive days of treatment compared to the control group.

6—No morphological damage observed in the rat organs as compared to the control group.

7—Microstructure of liver and kidney of rats: There was no significant difference compared with the biological control group after 21 days of continuous treatment on the skin damage model.

1. A handheld cold plasma device, comprising:
a power source;
an electronic control circuit board connected to a high-frequency high-voltage transformer;
a user control panel;
an insulated handle housing covering the power source, electronic control circuit, and high-frequency high-voltage transformer and the user control panel to form a monoblock;
a plasma generating unit containing an active electrode; and
a passive electrode on the outside of the handle housing configured to be connected to a neutral wire of the device.

2. The handheld cold plasma device of claim 1, wherein the high-frequency high-voltage transformer is capable of converting an electrical pulse of the control circuit into a damped sinusoidal pulse characterized by a first half sinusoidal having a higher amplitude and a narrower width and a second half sinusoidal having a smaller amplitude and a wider width to create capacitive balance and generate a polarized plasma.

3. The handheld cold plasma device of claim 2, wherein the high-frequency high-voltage transformer generates high voltage pulses according to the Ruhmkorff ferromagnetic resonance principle.

4. The handheld cold plasma device of claim 3, wherein the high-frequency high-voltage transformer is comprises a primary coil winding and a secondary coil winding coaxially together on a same ferromagnetic core, wherein the secondary coil winding is wound into several parts separated by insulating walls.

5. The handheld cold plasma device of claim 4, wherein the high-frequency high-voltage transformer is configured to produce damped sinusoidal electrical pulses with high oscillation frequencies above 20 kHz, and an output voltage amplitude between 1 kV and 30 kV.

6. The handheld cold plasma device of claim 5, where the passive electrode is in contact with the user in use and is connected to the neutral wire of the high-frequency high-voltage transformer system at the primary section by a capacitor.

7. The handheld cold plasma device of claim 6, wherein the passive electrode comprises a first layer of conductive material partially covering an area of the handle housing on an exterior of the insulated handle housing and a second layer of conductive material inside the insulated handle housing, the first layer and the second layer being parallel to one another and forming a capacitor.

8. The handheld cold plasma device of claim 7, wherein the electronic control circuit board is configured to change a polarization sign of the plasma by changing a sign of the generated electrical pulse.

9. The handheld cold plasma device of claim 1, wherein the electronic control circuit board is configured to connect with a smart device by a wired or wireless connection to exchange information related to treatment with the smart device.

10. The handheld cold plasma device of claim 9, wherein the electronic control circuit board is programmed to generate sequence of pulses in accordance with a predetermined program installed on the device or conveyed to the device from the smart device via the wired or wireless connection.

11. The handheld cold plasma device of claim 1, wherein the plasma generating unit is removable and operates according to a direct plasma principle.

12. The handheld cold plasma device of claim 1, wherein the plasma generating unit is removable and operates according to a principle of dielectric barrier discharge with a floating electrode.

13. The handheld cold plasma device of claim 1, wherein the high-frequency high-voltage transformer generates high voltage pulses according to the Ruhmkorff ferromagnetic resonance principle.

14. The handheld cold plasma device of claim 1, wherein the high-frequency high-voltage transformer is comprises a primary coil winding and a secondary coil winding coaxially together on a same ferromagnetic core, wherein the secondary coil winding is wound into several parts separated by insulating walls.

15. The handheld cold plasma device of claim 1, wherein the high-frequency high-voltage transformer is configured to produce damped sinusoidal electrical pulses with high oscillation frequencies above 20 kHz, and an output voltage amplitude between 1 kV and 30 kV.

16. The handheld cold plasma device of claim 15, wherein the high-frequency high-voltage transformer is configured to produce damped sinusoidal electrical pulses with high oscillation frequencies above 1000 kHz.

17. The handheld cold plasma device of claim 15, wherein the high-frequency high-voltage transformer is configured to produce an output voltage amplitude between 5 kV and 10 kV.

18. The handheld cold plasma device of claim 1, where the passive electrode is in contact with the user in use and is connected to the neutral wire (mass) of the high-frequency high-voltage transformer system at the primary section by a capacitor.

19. The handheld cold plasma device of claim 18, wherein the passive electrode comprises a first layer of conductive material partially covering an area of the handle housing on an exterior of the insulated handle housing and a second layer of conductive material inside the insulated handle housing, the first layer and the second layer being parallel to one another and forming a capacitor.

20. The handheld cold plasma device of claim 1, wherein the electronic control circuit board is configured to change a polarization sign of the plasma by changing a sign of a generated electrical pulse.
